(19) United States
(12) Patent Application Publication
(10) Pub. No.: US 2021/0251548 A1
(43) Pub. Date: Aug. 19, 2021

(54) LOW-POWER HEART BEAT DETECTION CIRCUIT ARCHITECTURE FOR BODY HEAT POWERED SENSING

(71) Applicant: Oregon State University, Corvallis, OR (US)

(72) Inventors: Soumya Bose, Corvallis, OR (US); Boyu Shen, Corvallis, OR (US); Matthew Johnston, Corvallis, OR (US)

(73) Assignee: Oregon State University, Corvallis, OR (US)

(21) Appl. No.: 17/169,136

(22) Filed: Feb. 5, 2021

Related U.S. Application Data
(60) Provisional application No. 62/977,063, filed on Feb. 14, 2020.

(51) Int. Cl. A61B 5/308; A61B 5/00

(52) U.S. Cl. CPC A61B 5/308; A61B 2560/0252; A61B 2560/0214; A61B 5/7225

(57) ABSTRACT

A battery-less heartbeat monitoring system that operates continuously using energy harvested from a low-grade heat source or small thermal gradient, such as human body heat and the gradient that exists between skin and the ambient environment in most circumstances.

### CLAIM FOR PRIORITY

This application is a continuation of, and claims the benefit of priority to U.S. Provisional Patent Application No. 62/977,063, filed on Feb. 14, 2020, titled “LOW-POWER HEART BEAT DETECTION CIRCUIT ARCHITECTURE FOR BODY HEAT POWERED SENSING,” and which is incorporated by reference in entirety.

### GOVERNMENT SUPPORT

This invention was made with government support under Award No. R21 DE027170 awarded by National Institutes of Health. The government has certain rights in the invention.

### BACKGROUND

Wearable devices are expanding beyond consumer and entertainment applications, including continuous monitoring of vital signs for medical diagnostics, due to extended ambulatory measurement capabilities compared to fixed clinical environments. Continuous long-term use is difficult for battery-powered devices in a wearable form-factor, whereas ambient energy harvesting promises power autonomy to enable indefinite lifetime for true battery-less devices. However, accurate vitals measurement under real-world motion conditions is energy intensive, making it difficult to provide reliable and medically relevant data using typical micro-watt power levels available from energy harvesting.

### BRIEF DESCRIPTION OF THE DRAWINGS

FIG. 1 illustrates block level architecture of the body heat powered, motion-resilient heartbeat monitoring system-on-chip, in accordance with some embodiments of the present disclosure.

FIG. 2 illustrates a set of plots showing adaptive threshold voltage generation and tracking technique with double-sided comparison for robust heartbeat detection in the presence of motion artifacts, in accordance with some embodiments of the present disclosure.

FIG. 3 illustrates an apparatus of adaptive threshold generation utilizing pulse width locked loop (PWLL), in accordance with some embodiments of the present disclosure.

FIG. 4 illustrates a state machine for heartbeat detection logic, in accordance with some embodiments.

FIG. 5 illustrates a single-inductor self-starting boost converter architecture for autonomous thermoelectric energy harvesting, in accordance with some embodiments.

FIG. 6 illustrates a fully-passive switched capacitor low-pass filter in the analog frontend (AFE), in accordance with some embodiments.

FIG. 7 illustrates a set of plots showing measured heartbeat detection accuracy using double-sided comparison against adaptive threshold voltages, and comparison with a fixed threshold approach, in accordance with some embodiments of the present disclosure.

FIG. 8 illustrates a set of plots showing measured start-up transients of power rails during cold-start, initialization of the AFE and VREFs; and harvested power utilization at minimum TEG voltage, in accordance with embodiments of the present disclosure.

FIG. 9 illustrates a flowchart of a method for determining a heartbeat, in accordance with some embodiments.

FIG. 10 illustrates a measurement setup for heartbeat detection with two-electrode electrocardiogram (ECG), in accordance with embodiments of the present disclosure, and performance comparison with prior work.

FIG. 11 illustrates a die micrograph of one exemplary embodiment of the SoC (system-on-chip) in a 0.18 μm Complementary Metal Oxide Semiconductor (CMOS) process technology node.

### DETAILED DESCRIPTION

Thermoelectric energy harvesting from a low-grade heat source like human body heat provides continuous availability across indoor, outdoor, light, and dark conditions. However, limited power at the low voltage (e.g., less than 100 mV) of a small TEG (thermoelectric generator) at typical skin-to-ambient temperature gradients (ΔT=1-2° C.) requires efficient DC-DC boost conversion.

Some embodiments disclose a battery-less heartbeat monitoring system that operates continuously using merely energy harvested from a low-grade heat source or small thermal gradient, such as human body heat and the gradient that exists between skin and the ambient environment in most circumstances. In some embodiments, a motion resilient heartbeat (HB) detection apparatus is provided that adjusts high and/or low reference voltage levels in accordance with pulse widths of electrocardiograph (ECG) signals. To mitigate the effect of motion artifacts, the apparatus uses pulse width locked loop (PWLL) to generate an adaptive threshold voltage for heartbeat detection. The apparatus uses low-power analog processing of an ECG signal to provide robust rejection of both slow and fast motion artifacts, enabling accurate detection of heartbeats in an ambulatory environment.

The apparatus looks for fast rising and falling edges in the ECG signal (or differentiated ECG signal, herein ECGD) to determine a valid heartbeat from an invalid heartbeat. An invalid heartbeat may have a signal signature such as an overshoot or undershoot. A valid heartbeat has a rising signal immediately followed by a falling signal, or vice versa. If there is a time gap between the rising signal and falling signal (or vice versa) that is greater than a threshold (e.g., programmable or fixed threshold), then the heartbeat is considered invalid. In some embodiments, the apparatus dynamically adjusts the high and/or low thresholds as the pulse width of the ECG signal varies, and as such the apparatus captures all possible heartbeats in the ECG signal. The logic of the apparatus then removes any artifacts from the possible heartbeats to determine the valid heartbeat from an invalid heartbeat, and outputs a stream of valid heartbeats free from artifacts.

The apparatus of various embodiments is configured for a wearable device. A wearable device is a device that can be worn as an accessory, embedded in clothing, implanted in the user's body, or even tattooed on the skin. The wearable device herein is a hands-free gadget for detecting heartbeat and which harvests its own power. Data from the wearable device is transmitted to any processor or server for further processing.

In some embodiments, a system-on-chip (SoC) is provided which comprises a first circuitry (e.g., energy harvesting and power management unit) to generate a first power supply voltage on a first rail and a second power supply voltage on a second rail, w herein the first power supply voltage is generated from energy harvested from a living body, and wherein the second power supply is generated using the first power supply voltage as input supply. In some embodiments, the SoC comprises a second circuitry (e.g., an analog frontend) to detect an electrocardiogram (ECG) signal from the living body, wherein the second circuitry operates on the second power supply. In some embodiments, the SoC comprises a third circuitry (e.g., motion resilient heartbeat detection circuitry) to determine a heartbeat from the ECG signal, wherein the third circuitry operates on the first power supply. In some embodiments, the SoC comprises a fourth circuitry (e.g., a near field communication transmitter) to transmit the heartbeat via an antenna. The SoC is powered by a low-voltage, self-starting DC-DC boost converter, enabling a minimum number of off-chip comments. Other components include a portable thermoelectric generator (TEG), an inductor, and a couple storage capacitors to enable autonomous, efficient body-heat energy harvesting in a wearable form factor.

In some embodiments, the first circuitry comprises: a DC-DC boost converter to generate the first power supply voltage DV_{DD}, and a low dropout (LDO) regulator to generate the second power supply voltage AV_{DD} from the first power supply voltage. In some embodiments, the second circuitry comprises: a low-noise amplifier (LNA) to receive signals from two sensors on the living body; a differentiator coupled to the low-noise amplifier; and a filter coupled to the differentiator, wherein an output of the filter is the ECG signal.

In some embodiments, the third circuitry comprises a first electrical loop comprising a first comparator to receive a signal representative of an electrocardiography signal or its derivative and to compare it with a first adjustable reference. The third circuitry further comprises a second electrical loop comprising a second comparator to receive the signal representative of an electrocardiography signal or its derivative and to compare it with a second adjustable reference, wherein the first adjustable reference has a voltage level higher than a voltage level of the second adjustable reference. In various embodiments, the third circuitry comprises logic to detect or classify a heartbeat in accordance with an output of the first and second comparators.

In some embodiments, the first electrical loop comprises a first pulse width detector (PWD) coupled to an output of the first comparator, wherein the first PWD is to generate a first Up and/or Down signal indicative of a first pulse width of the output of the first comparator with reference to a third reference, wherein the third reference is adjustable by software and/or hardware. In some embodiments, the third circuitry comprises: a first capacitor; and a first charge pump coupled to the first PWD, wherein the first charge pump is to sink or source current into the first capacitor in accordance with the first Up and/or Down signals, wherein a change on the first capacitor translates to the first adjustable reference.

In some embodiments, the second electrical loop comprises a second PWD coupled to an output of the second comparator, wherein the second PWD is to generate second Up and/or Down signals indicative of a second pulse width of the output of the second comparator with reference to a fourth reference, wherein the fourth reference is adjustable by software and/or hardware. In some embodiments, the third circuitry comprises: a second cap and a second charge pump coupled to the first MD, wherein the second charge pump is to sink or source current into the second capacitor in accordance with the second Up and/or Down signals, wherein a change on the second capacitor translates to the second adjustable reference. In some embodiments, the logic comprises fifth circuitry to determine whether the first and second outputs indicate a signal pattern with high and low pulses without a time gap, and if such signal pattern is observed, detect the heartbeat as a valid heartbeat, otherwise indicate an invalid heartbeat.

There are many technical effects of various embodiments. For example, the SoC of various embodiments is configured for a wearable device and detects a heartbeat of a living body (e.g., human body) without a power source such as a battery. The power is internally generated by the SoC by energy harvesting means. A heartbeat detection scheme is provided in the SoC that identifies heartbeat from other motions, and thus provides an accurate heartbeat information. Other technical effects will be evident from the various embodiments and figures.

In the following description, numerous details are discussed to provide a more thorough explanation of embodiments of the present disclosure. It will be apparent, however, to one skilled in the art, that embodiments of the present disclosure may be practiced without these specific details. In other instances, well-known structures and devices are shown in block diagram form, rather than in detail, in order to avoid obscuring embodiments of the present disclosure.

Note that in the corresponding drawings of the embodiments, signals are represented with lines. Some lines may be thicker, to indicate more constituent signal paths, and/or have arrows at one or more ends, to indicate primary information flow direction. Such indications are not intended to be limiting. Rather, the lines are used in connection with one or more exemplary embodiments to facilitate easier understanding of a circuit or a logical unit. Any represented signal, as dictated by design needs or preferences, may actually comprise one or more signals that may travel in either direction and may be implemented with any suitable type of signal scheme.

Throughout the specification, and in the claims, the term “connected” means a direct connection, such as electrical, mechanical, or magnetic connection between the things that are connected, without any intermediary devices.

Here, the term “analog signal” is any continuous signal for which the time varying feature (variable) of the signal is a representation of some other time varying quantity, i.e., analogous to another time varying signal.

Here, the term “digital signal” is a physical signal that is a representation of a sequence of discrete values (a quantified discrete-time signal), for example of an arbitrary bit stream, or of a digitized (sampled and analog-to-digital converted) analog signal.

The term “coupled” means a direct or indirect connection, such as a direct electrical, mechanical, or magnetic connection between the things that are connected or an indirect connection, through one or more passive or active intermediary devices.

The term “adjacent” here generally refers to a position of a thing being next to (e.g., immediately next to or close to with one or more things between them) or adjoining another thing (e.g., abutting it).

The term “circuit” or “module” may refer to one or more passive and/or active components that are arranged to cooperate with one another to provide a desired function.

The term “signal” may refer to at least one current signal, voltage signal, power signal, magnetic signal, or data/clock signal. The meaning of “a,” “an,” and “the” include plural references. The meaning of “in” includes “in” and “on.”

The term “scaling” generally refers to converting a design (schematic and layout) from one process technology to another process technology and subsequently being reduced in layout area. The term “scaling” generally also refers to downsizing layout and devices within the same technology node. The term “scaling” may also refer to adjusting (e.g., slowing down or speeding up—i.e. scaling down, or scaling up respectively) of a signal frequency relative to another parameter, for example, power supply level. The terms “substantially,” “close,” “approximately,” “near,” and “about,” generally refer to being within +/−10% of a target value.

Unless otherwise specified, the use of the ordinal adjectives “first,” “second,” and “third,” etc., to describe a common object, merely indicate that different instances of like objects are being referred to and are not intended to imply that the objects so described must be in a given sequence, either temporally, spatially, in ranking or in any other manner.

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C).

The terms “left,” “right,” “front,” “back,” “top,” “over,” “under,” and the like in the description and in the claims, if any, are used for descriptive purposes and not necessarily for describing permanent relative positions.

It is pointed out that those elements of the figures having the same reference numbers (or names) as the elements of any other figure can operate or function in any manner similar to that described but are not limited to such.

For purposes of the embodiments, the transistors in various circuits and logic blocks described here are metal oxide semiconductor (MOS) transistors or their derivatives, where the MOS transistors include drain, source, gate, and bulk terminals. The transistors and/or the MOS transistor derivatives also include Tri-Gate and Fin Field Effect Transistors (FETs), Gate All Around Cylindrical Transistors, Tunneling FET (TFET), Square Wire, Rectangular Ribbon Transistors, ferroelectric FET (FeFETs), vertical MOSFETs, or other devices implementing transistor functionality like carbon nanotubes or spintronic devices. In vertical MOSFETs, drain and source terminals are not symmetrical. For typical MOSFET source and drain terminals are symmetrical i.e., are identical terminals, and are interchangeably used here. A TFET device, on the other hand, has asymmetric Source and Drain terminals. Those skilled in the art will appreciate that other transistors, for example, Bi-polar junction transistors (BJT PNP/NPN), BiCMOS, CMOS, etc., may be used without departing from the scope of the disclosure.

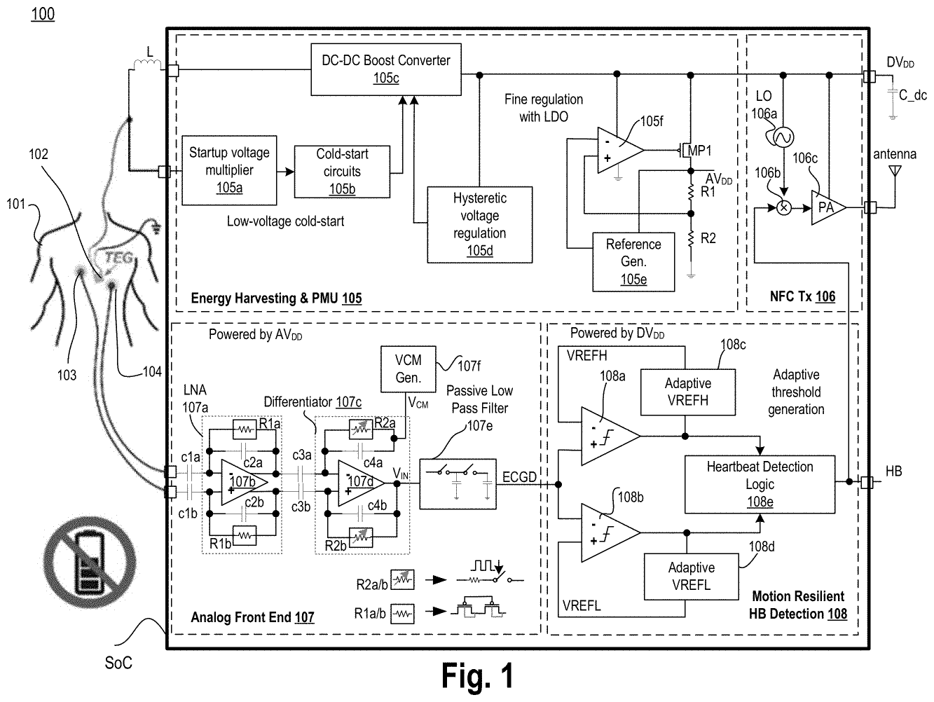

FIG. 1 illustrates block level architecture 100 of the body heat powered, motion-resilient heartbeat monitoring system-on-chip in accordance with some embodiments of the present disclosure. Architecture 100 comprises an inductor L, a body 101, centimeter-scale thermoelectric generator (TEG) 102, first sensor 103, second sensor 104, a system-on-chip (SoC) or processor including an energy harvesting and power management unit (PMU) 105, near field communications (NFC) transmitter (Tx) 106, Analog Front-End (AFE) 107, and Motion Resilient Heartbeat (HB) detection circuitry 108, an antenna and decoupling capacitor C_dc. In some embodiments, the inductor can be on-die (e.g., part of processor or SoC) or off-die (e.g., outside of processor or SoC, on package, or outside of the package). In some embodiments, the antenna and/or coupling capacitor C_dc are on-package of the SoC, or outside of the package of the SoC.

In some embodiments, energy harvesting and PMU 105 comprises startup voltage multiplier 105a, cold-start circuits 105b, DC-DC boost converter 105c, hysteretic voltage regulation 105d, low dropout (LDO) regulator having amplifier 105f, reference generator 105e, one or more transistors MP1, and voltage divider network comprising resistors R1 and R2.

In some embodiments, DC-DC converter 105c converts a source of direct current (DC) from one voltage level to another. DC-DC converter 105c can be linear or switched. A linear DC/DC converter uses a resistive voltage drop to create and regulate a given output voltage. Conversely, a switched-mode DC-DC converts by storing the input energy periodically and then releasing that energy to the output at a different voltage. In some embodiments, DC-DC converter 105c self-starts with a low voltage (e.g., as low as 60 mV) from a centimeter-scale thermoelectric generator (TEG) 102 and delivers power for the operation of the SoC. In some embodiments, DC-DC boost converter 105c is operated with maximum power point tracking (MPPT) that enables it to draw the maximum available power from TEG 102 by matching its input impedance with that of the source. The output of DC-DC converter is DV_{DD}, which is the power supply for the SoC. Here, node names and signal names are interchangeably used. For example, DV_{DD} may refer to power supply rail, power supply voltage or current, depending on the context of the sentence.

In some embodiments, low-voltage boost converter 105c is designed to operate in discontinuous conduction mode (DCM), conducive for power-constrained applications. In some embodiments, converter 105c self-starts with the aid of a low-voltage, on-chip voltage multiplier and integrated cold-start circuits. A single-inductor boost converter topology with low-voltage, on-chip cold-start eliminates additional energy requirements for start-up in a compact form-factor. The switching frequency of DC-DC converter 105c is tuned once to match its input impedance with the fixed TEG source resistance to ensure MPPT at the input.

Following cold-start, the output voltage (DV_{DD}) is regulated at, for example, 1.5V by a voltage-mode hysteresis loop using two on-chip thresholds generated with low-power reference circuits. Hysteretic regulation causes voltage ripple on DV_{DD} and makes it unsuitable for powering a low-noise AFE directly. In some embodiments, startup voltage multiplier 105a is a low voltage circuit that boosts an input voltage initially and powers the startup circuit 105b to activate DC-DC boost converter from cold-start. Following cold-start, the output DV_{DD} of DC-DC boost converter 105c reaches a higher voltage (e.g., 1.5 V) and is regulated by a hysteretic on-off voltage regulator 105d. to provide a clean supply for analog-signal-conditioning circuits, DVDD is further regulated to AV_{DD} (e.g., 1.2V).

In some embodiments, a low-I_{Q} linear regulator such as a low dropout (LDO) regulator with high power supply rejection ratio (PSRR) (e.g., greater than 50 dB) is designed, including an embedded nanowatt bandgap reference to generate a less noisy regulated voltage (AV_{DD}) of, for example 1.2 V, for powering AFE 107. In some embodiments, DV_{DD} powers digital blocks directly and is connected to an off-chip decoupling capacitor (e.g., greater than 1 μF) to support higher switching currents. In some embodiments, storage capacitors (e.g., internal on-die or external off-die) are used to support power delivery in the SoC. One of those capacitors is used at the output of TEG 102 and another capacitor (e.g., 1 micro Farad) is used at the boosted voltage output of converter 105c to support digital circuit switching currents.

In some embodiments, the LDO regulator uses DV_{DD} as an input power supply and generates a regulated supply AV_{DD} from DV_{DD}. Comparator or amplifier 105f of the LDO compares the voltage on the AV_{DD} supply rail or a divided version of the voltage on the AV_{DD} supply rail with a reference voltage. The reference voltage may be generated by any suitable source such as a resistor ladder, bandgap circuit 105e. The voltage/current on the AV_{DD} supply rail is used to power AFE 107, while the output DVDD of DC-DC Boost Converter 105c is used to power NFC Tx 106 and HB detection circuitry 108.

In some embodiments, signal processing analog front-end (AFE) 107 amplifies and shapes a two-electrode electrocardiogram (ECG) signal from sensors 103 and 104 for feature extraction. Sensors 103 and 104 can be gel electrodes. In some embodiments, AFE 107 comprises low noise amplifier (LNA) 107a, differentiator 107c, common mode voltage generator (VCM) 107f, and filter 107e. In some examples, VCM 107f generates AV_{DD}/2, common mode voltage level. LNA 107a is coupled to sensors 104 and 103 via coupling capacitors C1a and C1b, respectively. A simplified version of LNA 107a is illustrated here. However, any suitable low-voltage LNA can be used for LNA 107a. LNA 107a is an amplifier that amplifies a very low-power signal on sensors 103 and 104 without significantly degrading its signal-to-noise ratio. LNA 107a comprises a differential amplifier 107b with inputs coupled to the outputs via resistor-capacitor (RC) network comprising capacitors C2a, C2b, and resistors R1a, R1b coupled as shown. In some embodiments, the capacitances of C2a, C2b are adjustable. In some embodiments, resistors R1a, R1b are implemented as discrete resistors or one or more transistors configured as resistors. The differential output of LNA 107a is coupled to differentiator 107c via coupling capacitors C3a and C3b as shown. In some embodiments, the range of capacitances of capacitors C1a and C1b is a range of 10 pF to 50 pF. In some embodiments, capacitances of capacitors C2a and C2b is a fraction of capacitances of capacitors C1a and C1b. For example, capacitances of capacitors C2a and C2b is 1/10^{th} of capacitances of capacitors C1a and C1b to provide a gain of 10 for the first stage. In some embodiments, capacitances of capacitors C3a and C3b is in a range of 3 pF to 7.5 pF. In some embodiments, resistance of resistors R1a and R1b is greater than 400 GOhm.

In some embodiments, differentiator 107c comprises amplifier 107d (e.g., differential-to-single-ended amplifier). One input (e.g., negative input) of amplifier 107d receives a filtered common voltage from common mode voltage (VCM) generator 107f. The filter comprises resistor R2a and capacitor C4a. In some embodiments, the resistor R2a comprises a switched resistor circuitry (e.g., a switch coupled in series with the resistor). The switch is controlled by a duty-cycled clock, CK_{D}, which is derived from a clock CLK. In some embodiments, the other input (e.g., positive input) of amplifier 107d is coupled to the output of amplifier 107d via capacitor C4b and resistor R2b. In some embodiments, the resistor R2b comprises a switched resistor circuitry. In some examples, the range of resistances of resistors R2a and R2b is greater than 100 GOhm. In some example, the capacitance of capacitors C4a and C4b is in a range of 100 fF to 500 fF. In some embodiments, the output of differentiator 107c is filtered by filter 107e to generate the ECGD signal (electrocardiogram signal), which is then processed by HB detection circuitry 108 to extract the heartbeat (HB).

In some embodiments, the output ECGD of AFE 107 is compared against two independent adaptive threshold voltages for accurate heartbeat detection in the presence of motion artifacts. The outputs of the double-sided comparison block are further used by digital backend circuits for motion-artifact-resilient heartbeat detection.

In some embodiments, HB detection circuitry 108 applies an adaptive threshold generation and tracking scheme that detects heartbeats (from the ECGD) with high accuracy in the presence of motion artifacts. In some embodiments, HB detection circuitry 108 comprises first comparator or amplifier 108a, second comparator or amplifier 108b, high-reference voltage (VREFH) generator 108c, low-reference voltage (VREFL) generator 108d, and heartbeat detection logic 108e. Comparator 108a compares ECGD with VREFH while comparator 108b compares ECGD with VREFL. The outputs of comparators 108a and 108b are received by heartbeat detection logic 108e that isolates the heartbeat from any noise in ECGD.

In some embodiments, the detected heartbeats (HB) are wirelessly transmitted using near field communications (NFC) transmitter 106. In one example, the detected heartbeats are transmitted wirelessly in 1-ms bursts using NFC via an on-off keying (OOK) modulation scheme. NFC Tx 106 comprises local oscillator (LO) 106a, mixer 106b, and power amplifier (PA) 106c. The output of PA 106c is coupled to an antenna. LO 106a may generate a carrier frequency which mixes the HB signal to move it to a higher or lower frequency band. The output of mixer 106b may be an unconverted signal which is amplified by PA 106c for transmission via the antenna. In some embodiments, the whole system operates with a minimum TEG voltage of, for example 20 mV, and a low harvester input power of, for example, 20 μW.

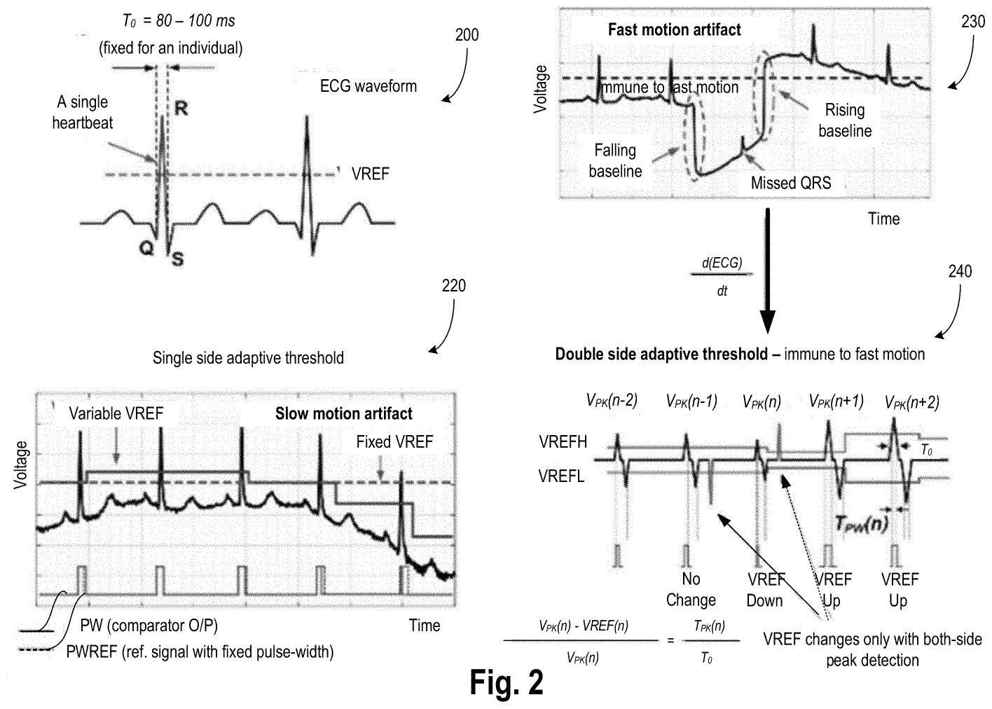

FIG. 2 illustrates a set of plots 200, 220, 230, and 240 showing the adaptive threshold voltage generation and tracking technique with double-sided comparison for robust heartbeat detection in the presence of motion artifacts in accordance with some embodiments of the present disclosure. The apparatus of various embodiments results in low-power, motion-resilient feature extraction from ECG, as illustrated in FIG. 2, where a power-efficient digital loop is used to generate threshold voltages adaptive to motion, including normal human motions like walking, stretching, or running.

Heartbeats occur at QRS peaks in an ECG waveform. As such, a heartbeat can be extracted from the QRS complex by comparing a clean ECG signal against a fixed voltage threshold (VREF). The QRS complex is the combination of three of the graphical deflections seen on a typical electrocardiogram (EKG or ECG). It is usually the central and most visually obvious part of the tracing; in other words, it's the main spike seen on an ECG line. While a clean ECG signal is easy to record in a clinic, this is more difficult for a wearable sensor subject to motion artifacts such as respiration and other bodily motions.

An AC-coupled low noise amplifier (LNA) and analog filter in the front-end can remove DC offset and fixed-frequency line noise, but this approach cannot remove arbitrary motion artifacts. Motion artifacts can be characterized by a sudden rise or fall of the ECG baseline; as the duration of the QRS complex is fairly consistent for an individual, comparison with a fixed VREF will result in a comparator output signal, PW (pulse width) with varying pulse widths (T_{PW}), implying motion due to shifted baseline. Comparison of PW against a reference signal, PW_{REF}, with fixed pulse-width and generation of a voltage, ΔV_{PK}, proportional to the pulse width error (ΔT_{PW}=T_{PW}−T_{PWREF}) can be utilized to update VREF in the next cycle; using this approach, the voltage shifts in QRS peak value caused by motion are fed back to adjust the threshold voltage. (Note, here T_{PWREF} and T_{REF}, refer to the same reference).

However, this VREF update rate is limited by heart rate, whereas sudden movements may cause fast rise or fall of the ECG baseline that will not be captured by the PW (pulse width) comparison. To address this challenge, differentiator 107c is used to detect fast edges, realized as a high-pass filter in the AFE 107 (FIG. 1). It also produces a bipolar peak for each occurrence of a QRS complex (FIG. 2); detection of both peaks validates a true heartbeat, distinct from one-sided peaks due to fast motion artifacts. In some embodiments, for robust motion-resilient heartbeat detection, adaptive threshold voltages (VREFL and VREFH) are generated on both high and low sides to sense the change in the pulse-width of the differentiator output (ECGD), rather than on single-sided QRS; as a result, the double-sided comparison makes the heartbeat detection scheme adaptive to both slow and fast motion artifacts.

As discussed herein, motion information is extracted directly from the ECG signal. The QRS duration To is relatively fixed (e.g., 80 to 100 ms) for an individual. As shown in plots 220 and 240, with width of this QRS complex at a known voltage reference can be used to track baseline drift and to generate motion-adaptive threshold for heartbeat detection. Approximating a single QRS complex as a triangle, for the triable similarity, the following is obtained:

$$\frac{{V}_{REF} ( n )}{{V}_{PK} ( n )} = 1 - \frac{{T}_{P W} ( n )}{{T}_{0}} \qquad ( 1 )$$

where V_{PK}(n) is the peak voltage, V_{REF}(n) is the reference voltage, and T_{PW}(n) is the pulse width of the comparator output V_{CMP} at the nth QRS complex. In equation (1), the time-domain signal T_{PW}(n) carries the information of relative voltage position between V_{PK}(n) and V_{PK}(n+1). If T_{PW}(n) is compared with a fixed reference pulse width, T_{REF} (e.g., chosen based on desired steady-state V_{REF}), the pulse width error ΔT(n) and the proportional error voltage ΔV(n) can be used to update the reference voltage for the next cycle, V_{REF}(n+1). In some embodiments, the reference tracking is modeled as:

$${V}_{R E F} ( n + 1 ) - {V}_{R E F} ( n ) = K . [ {T}_{P W} ( n ) - {T}_{R E F} ] = K {T}_{0} . ⌈ 1 - \frac{{T}_{R E F}}{{T}_{0}} - \frac{{V}_{R E F} ( n )}{{V}_{P K} ( n )} ⌉ \qquad ( 2 )$$

where K is a conversion gain from time to voltage. To simplify equation (2), as well as to apply a linearized frequency-domain analysis, V_{PK}(n) is assumed to be a step function with a DC amplitude of V_{PK,DC}. A frequency-domain analysis provides:

$$H ( z ) = \frac{{V}_{R E F} ( z )}{{V}_{P K} ( z )} = \frac{{R}_{R E F} \cdot {V}_{0} / {V}_{PK , DC}}{z - ( 1 - {V}_{0} / {V}_{PK , DC} )} \qquad ( 3 )$$

where R_{REF}=1−T_{REF}/T₀ and V₀=K.T₀. Note that if V_{PK}(n) has higher frequency components, equation (3) may no longer hold, and the system feedback loop may be unstable. Form equation (3), the reference adapter H(z) has a DC gain R_{REF}, and hence, it can be used to adapt V_{REF}(n) with slow variations of V_{PK}(n).

The low-pass characteristic of H(z) serves the aim of reference tracking in the presence of baseline drift, and the tracking bandwidth can be adjusted by controlling K. In some cases, the update rate of this reference tracking is inherently limited to the heart rate, and it is not able to track abrupt transient artifacts as shown in plot 230. To address this challenge, differentiator 107c is used, where a QRS complex is distinguished by its bipolar slope compared with unipolar fast artifacts. As shown in plot 240, the detection of double-sided peaks validates a true heartbeat, district from one-sided peaks due to fast artifacts.

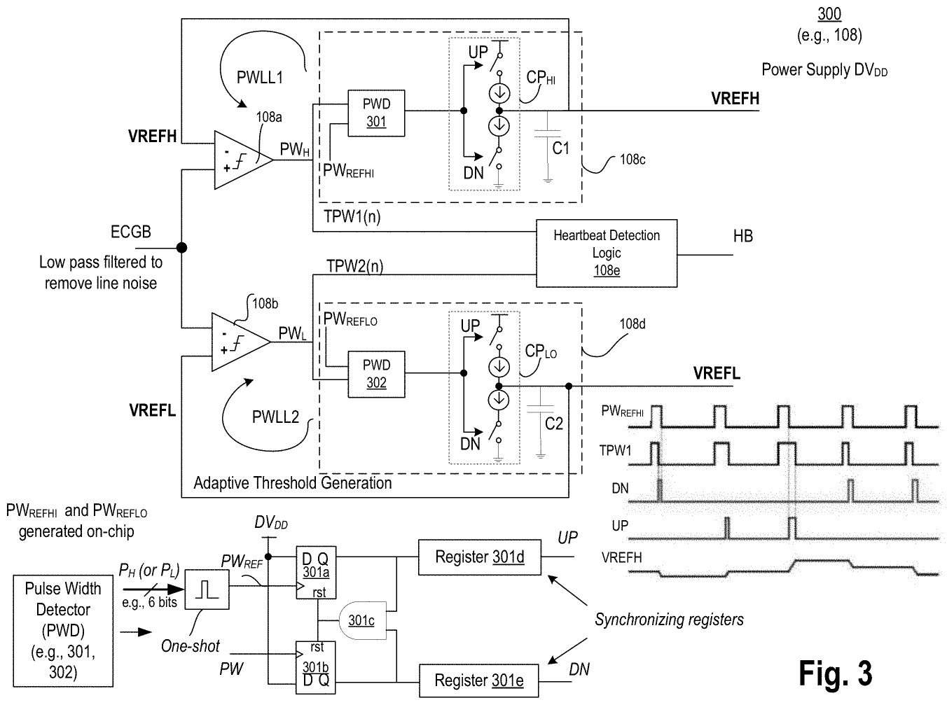

FIG. 3 illustrates a circuitry 300 of the adaptive VREF generation of circuitry 108 by means of a pulse-width locked loop (PWLL), in accordance with some embodiments. Circuitry 300 (e.g., 108) provides details of adaptive VREFH generator 108c and adaptive VREFL generator 108d. In some embodiments, adaptive VREFH generator 108c comprises pulse-width detector (PWD) 301, charge pump CP_{HI}, and first capacitor C1. PWD 301 compares pulse-width of the output of comparator 108a with that of a reference pulse PW_{REFHI} and generates Up and/or Dn signals. The Up signal may indicate that the output of comparator 108a is below PW_{REFHI} and so VREFH should be increased by CP_{HI}. The same concept applies to the down signal Dn. In some embodiments, charge pump CP_{HI} is to sink or source current into the first capacitor C1 in accordance with the Up and/or Down signals, wherein a change on the first capacitor translates to the adjustable reference VREFH. As such, operation principle of PWLL1 is similar to that of a phase-locked loop (PLL). Note, here PWLL compensates a difference in the pulse-widths which is equivalent to phase or frequency in a PLL.

In some embodiments, adaptive VREFL generator 108d comprises pulse-width detector (PWD) 302, charge pump CP_{LO}, and second capacitor C2. PWD 302 compares the output of comparator 108b with a reference PW_{REFLO} and generates Up and/or down (Dn) signals. The Up signal may indicate that the output of comparator 108b is below PW_{REFLO} and so VREFL should be increased by CP_{LO}. In some embodiments, charge pump CP_{LO} is to sink or source current into the second capacitor C1 in accordance with the Up and/or Down signals, wherein a change on the second capacitor C2 translates to the adjustable reference VREFL. The same concept applies to the down signal Dn. As such, the operating principle of PWLL2 is similar to that of a PLL.

Pulse width detector (PWD) architecture is similar to a phase detector in a PLL. One such architecture for PWD 301 or 302 is illustrated in FIG. 3. PWD 301 or 302 comprises samplers 301a and 301b (e.g., flip-flops), AND logic gate 301c, and synchronizing registers (e.g., latches or flip-flops) 301d and 301e. The data input for samplers 301a and 301b are coupled to DV_{DD}. The clock input of sampler 301a is coupled to PW_{REF} while the clock input of sampler 301b is coupled to PW. AND gate 301c resets the samplers based on the outputs of samplers 301a and 301b. Depending on the relative position of PW with respect to PW_{REF}, UP and DN (down) pulses are generated. The width of the pulses depends on the pulse-width difference between PW with respect to PW_{REF}.

TPW(n) (e.g., TPW1(n) and TPW2(n)) generated by comparison of ECGD with VREF(n) (e.g., VREFH and VREFL) is compared against TPWREF (e.g., PW_{REFHI} and PW_{REFLO}). Note, TPWREF is same as TREF. The difference ΔTPW(n) (output of PWD) is converted to a proportional voltage, ΔV(n) and added to VREF(n) (e.g., VREFH and VREFL) using a pulse-width detector (PWD) 301/302 and a charge pump (CP); the next comparison is against the updated VREF(n+1) (e.g., updated VREFH and VREFL).

In some embodiments, adaptive reference voltages VREFH and VREFL are independently generated by comparing the variation of the pulse widths of the comparator outputs TPW1(n) (also referred to as PW_{H}) and TPW2(n) (also referred to as PW_{L}) against that of the fixed reference pulses (PW_{REFHI} and PW_{REFLO}) to track the changes in both positive as well as negative peaks of ECGB (also referred here as V_{ECGB}). Note, PW_{H} and PW_{L} are the name of the signals at the comparator output. TPW1(n) and TPW2(n) are pulse widths of the nth pulse of signals PW_{H} and PW_{L}, respectively. The HB signal is generated when both peaks are detected, indicating a true heartbeat. This double-sided pulse width tracking makes the heartbeat detection adaptive to both slow and fast artifacts.

In various embodiments, circuitry 300 performs adaptive reference tracking for positive and negative peaks of VECGB using two independent PWLLs (PWLL1 or PWLL_{H} and PWLL2 or PWLL_{L}) with operates similar to a phase-locked loop. In some embodiments, comparators 108a and 108b are clocked comparators operated by CLK. Comparators 108a and 108b generates output signal TPW1(n) (also referred to as VCMP_{H}) and TPW2(n) (also referred to as VCMP_{L}), whose pulse width T_{PW} denotes the period when V_{ECGB} is above (for positive peak) or below (for negative peak) the present reference voltages (VREFH and VREFL). The rising edge of comparator output triggers a one-shot (PW_{REFHI} and PW_{REFLO}) with a fixed predefined pulse width T_{REF} (same as TPWREF).

The pulses are generated on-chip by individual timer blocks operated by CLK, and the pulse widths can be independently programmed using controls (e.g., 6-bit controls P_{H} and P_{L}). The two pulses PW and PW_{REF} are compared by a phase-frequency detector in either side. The corresponding error pulse is stored in a pulse delay block (e.g., registers 301d and 301e) and synchronized for both reference loops which serves two purposes:

First, detection logic 108e identifies a true heartbeat signal when dual-sided peaks are observed in the differentiated ECG signal. In this case, the reference voltage is adjusted to track the baseline. Detection of a sole positive or negative peak is classified as an artifact, and the reference is not altered.

Second, as the QRS during is long (e.g., approximately 100 ms), adjusting V_{REF} immediately may cause secondary comparator triggering. A pulse delay block is used to first quantize the pulse width error using the CLK (e.g., 1 kHz core clock, and the result is stored in a register. Once the heartbeat flag signal, HB is asserted, indicating a detected heartbeat, and the pulse delay block recreates the error pulse to steer the charge pump. As the QRS period is well-defined, an internal timer is used for timeout. If the interval between an error pulse and an HB signal exceeds a predefined expiration time, the register is reset to avoid false detection.

As described by equation (3), the reference tracking loop dynamics are controlled by the design parameters T_{REF} and K. T0 is set at 50 ms, which is half of the nominal QRS duration; V_{PK,DC} is chosen as 0-6V in one example for maximum input amplitude; and K is determined by convergence requirement:

$$K = \frac{{I}_{CP}}{{C}_{VREF}} < \frac{{V}_{PK , DC}}{{T}_{0}} \qquad ( 4 )$$

where I_{CP} is the charge pump current and C_{VREF} is the storage capacitor for V_{REF}. In one example, I_{CP} is designed as 500 pA, and V_{REF} is implemented as an adjustable capacitor bank providing 30 to 50 pF to control K. In some embodiments, transistors used in the charge pump are all selected as thick-oxide devices to minimize leakage of V_{REF} during intervals between successive heartbeats. In one example. the reference pulse width T_{REF} (n×T_{CLK}) can be programmed externally by a 6-bit register up to 64 ms with the 1-kHz clock to freely position the reference voltage. The given programmability also accommodates +/−10% variations of on-chip clock (CLK) frequency due to process corners, in accordance with some embodiments.

In some embodiments, although double-side peak detection uses dual PWLLs, additional power consumption is negligible due to the low VREF update rate. In addition to motion resilience, the technique of various embodiments eliminates the need to generate a process-voltage-temperature (PVT) tolerant fixed voltage reference. While an initial VREF (e.g., VREFH and VREFL) is still used to start the loop, this is generated with relaxed tolerances using a low-power, leakage-based reference generator, for example. VREFs on both sides (e.g., for 108c and 108d) are then self-trained within the first few ECG cycles and settle to values proportional to the QRS duration, removing the need for individual calibration. Detected heart beats in the form of, for example 1 ms, bursts are transmitted wirelessly by NFC Tx 106 (or any other suitable wireless means) using a simple on-off keying (OOK) modulation scheme (or any other suitable scheme).

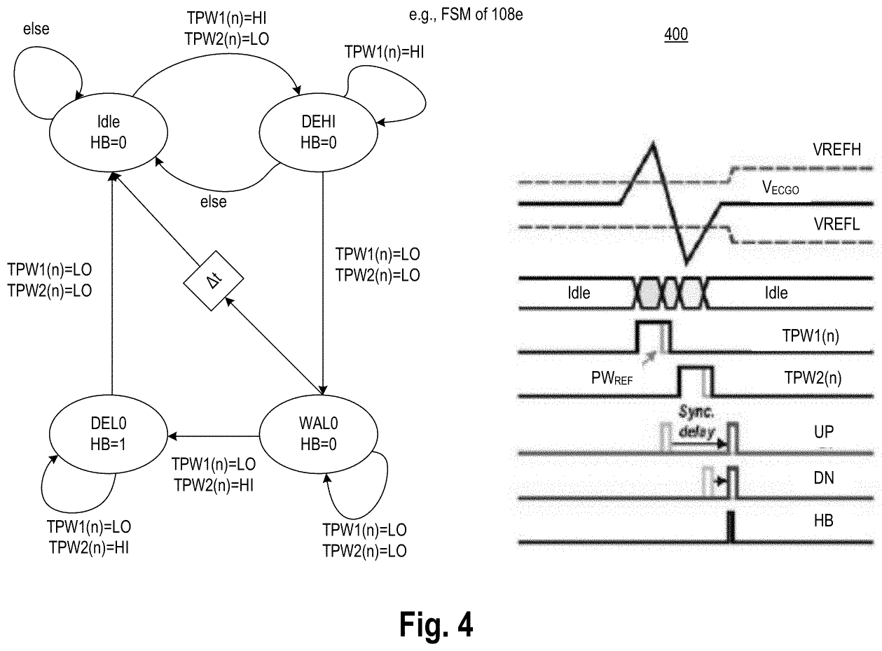

FIG. 4 illustrates state machine 400 for heartbeat detection logic, in accordance with some embodiments. Heartbeat detection logic 108e comprises a finite state machine (FSM) as shown in FIG. 4. The FSM has four states (e.g., idle, DEHI, WALO, DELO) that represent the idle state, positive peak detected, waiting for negative peak, and negative peak detected, respectively. The DELO state indicates a validated heartbeat detection, which triggers a one-ship pulse signal, HB, with a pulse width of one clock period. An internal timeout ensures that the detector does not indefinitely wait for the negative peak detecting comparator output during WALO, guaranteeing that a previous false triggering of the positive peak detecting comparator will not impact the future detections.

The positive and negative peak detection sequences leverage the structure of the QRS complex, where the slope is always positive and negative peak detection sequences leverage the structure of the QRS complex, where the slope is always positive and then negative, providing a robust double-sided heartbeat detection in the presence of noise and artifacts. Note that the detection scheme of various embodiments may have a delay of multiple clock cycles (e.g., n×1 ms) between the actual QRS and the HB pulse. In addition, the delay may vary from beat to beat based on the current position of VREFL relative to the negative peak of V_{ECGB}, resulting in a variation of the R-R interval. While this may limit utility for clinical R-R variation assessment, it will have a negligible impact for heart rate related applications. In addition, the variation may be less due to the slow tracking bandwidth of the scheme, and the error may be averaged output in the long-term heart rate measurement.

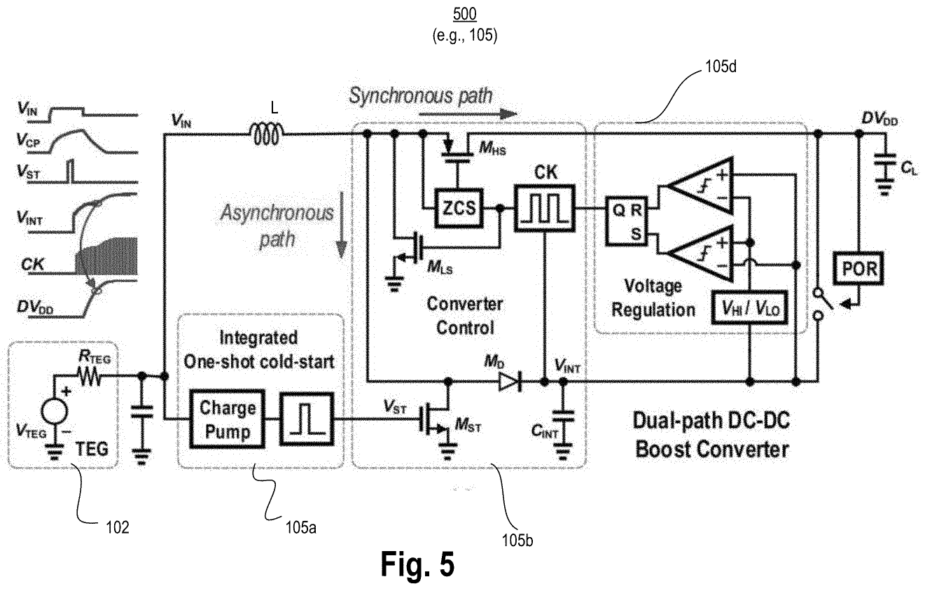

FIG. 5 illustrates a single-inductor self-starting boost converter architecture 500 (e.g., 105) for autonomous thermoelectric energy harvesting, in accordance with some embodiments. One challenge associated with the design of DC-DC boost converter 105c for harvesting thermal energy from body heating using TEG 102 are low-voltage cold-start and end-to-end power efficiency. To address that challenge, some embodiments use a single-inductor, dual-path boost converter topology 500. In architecture 500, the converter uses an asynchronous startup path comprising a low-side switch M_{ST} and a high-side p-type diode M_{D}. A charge-pump based on-chip voltage multiplier boost the TEG voltage during cold-start and provides power supply V_{CP} to the startup circuits for generating a one-shot.

An output of the multiplier ramps up to a voltage higher than, approx. 650 mV, the one-shot is asserted, and it assists in charging the inductor through M_{ST} with sufficient energy required to start a secondary oscillator clock CK. The falling edge of the one-shot triggers the asynchronous path to start boosting the input voltage. The intermediate boosted voltage V_{INT}, stored internally on a capacitor (e.g., 200 pF), supplies power to the oscillator and to a low-power voltage supervisor circuit. The output power of the voltage multiplier and the duration of the triggering pulse are used for the cold-start of the converter. In various embodiments, an n-stage (e.g., 20-stage) charge pump with initial cross-coupled complementary states for higher output power from the voltage multiplier is used. In some embodiments, the one-shot pulse width is reduced for faster and efficient cold-start.

Once V_{INT} crosses a threshold (e.g., 0.8 V), a secondary energy transfer path is enabled, which operates synchronously to transfer power to an output capacitor CL or C_dc (e.g., 1 micro-Farad). The synchronous path is operated in the discontinuous conduction mode (DCM) by synchronous control of the ON-time of the high-side switch MHS with the assistance of a digital zero-current sensing (ZCS) circuit. Both synchronous and asynchronous paths operate as time-multiplexed during startup until the output DV_{DD} reaches 1V. At this point, a power-on-reset (POR) circuit shorts V_{INT} and DV_{DD}, and the startup voltage multiplier and other cold-start circuits are disabled.

At the source resistance of TEG 102, R_{TEG}, is fixed, one-time tuning of the converter switching clock frequency is sufficient for maximum power point tracking (MPPT). In addition, a loss-optimized MPPT (LO-MPPT) technique is used to enhance end-to-end efficiency and available output power. The core clock frequency of the switch converter CK can be tuned during power-ON by controlling programmable bits to take care of any static variation of the source resistance. The voltage supervisor regulates DV_{DD} by means of an On-Off hysteretic regulation approach using two on-chip reference voltages. The single-inductor topology of the boost converter with integrated cold-start helps to achieve both low-voltage startup and sufficient output power at low input voltages, without using additional off-chip components.

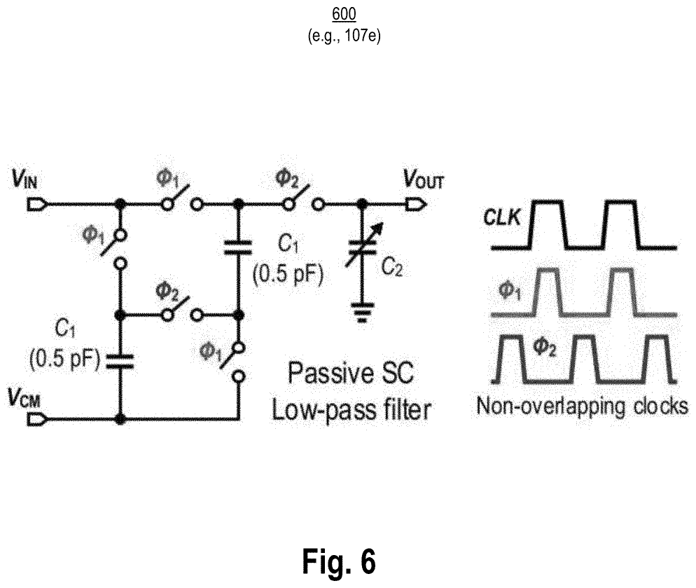

FIG. 6 illustrates a fully-passive switched capacitor low-pass filter 600 in the analog frontend (AFE), in accordance with some embodiments. In some embodiments, following differentiator 107c, first-order switched-capacitor low-pass filter 600 is provided. In some embodiments, filter 600 has a gain of 2. Filter 600 comprises switches controllable by clock phases Φ1 and Φ2, and capacitors C1 and C2 coupled as shown. In some embodiments, the switches are implemented as transistors (e.g., pass-gates). In some embodiments, capacitors are implemented as discrete capacitors, metal-insulator-metal (MIM) capacitors, ferroelectric capacitors, transistors configured as capacitors, etc. In one example, filter 600 provides approximately 20 dB attenuation to the residual 50 Hz or 60 Hz line noise in the differential ECG signal V_{ECGB}. The fully passive realization introduces no non-linearity and improves over active circuits for rail-to-rail operation, supply noise, and static power. In some embodiments, the load capacitor C2 is adjustable (e.g., from 1 pF to 8 pF) to achieve variable low-pass cutoff frequency.

In some embodiments, a clock CLK is generated on-chip (e.g., a 1 kHz clock) using a low-power oscillator. The clock serves as the core clock CLK for AFE 107 and heartbeat detection circuits. In some embodiments, in AFE 107, the clock CLK is used to generate sub-clock CLK_{D} which is utilized for duty-cycled resistance generation and/or the switched-capacitor operation of low-pass filter 600. The low-frequency nature of the ECG signal relaxes the requirement of a fast clock as well as the corresponding routing complexities.

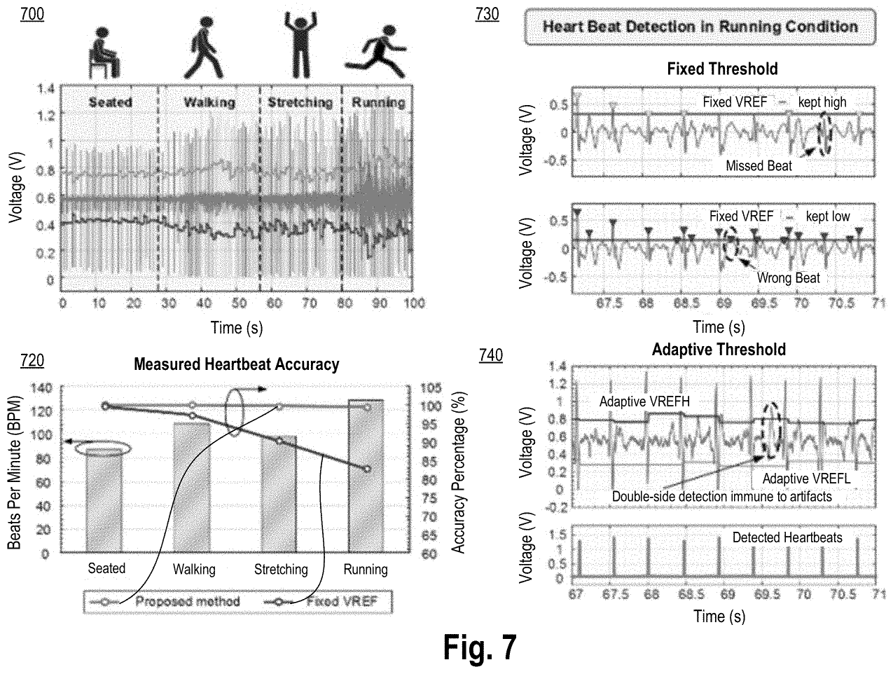

FIG. 7 illustrates a set of plots 700, 720, 730, and 740 that demonstrate motion artifact resilience in a two-electrode ECG measured from the prototype chip under different motion conditions. The adaptive threshold voltage generation scheme achieves high accuracy detecting heartbeats across motion types. For comparison, heartbeats are also measured in parallel by comparing the amplified raw ECG from AFE against a fixed threshold, which shows that conventional heartbeat detection is accurate while seated but deteriorates under body motion.

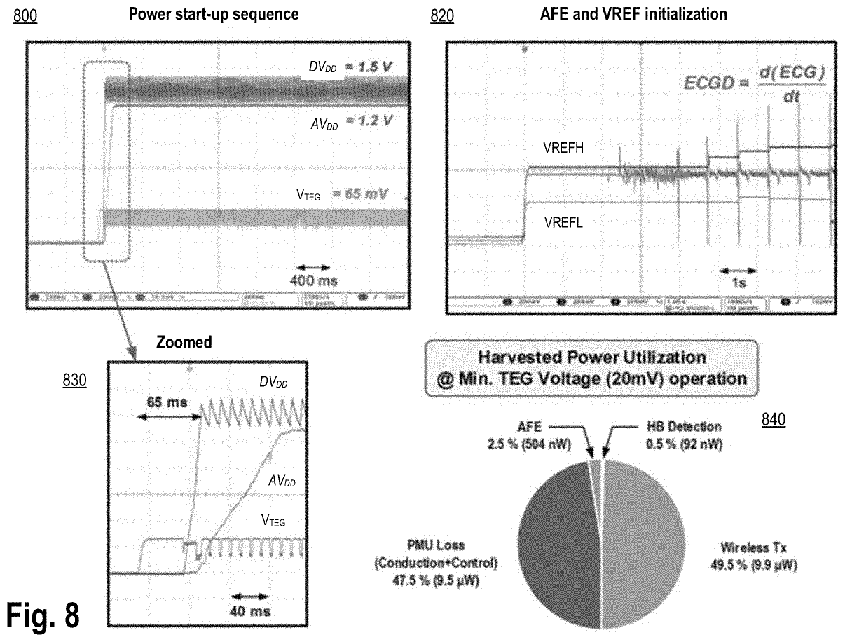

The battery-less system cold-starts with a minimum TEG voltage of 60 mV, using a minimum temperature difference of approximately 1.4° C. between warm surface (e.g., skin) and ambient environment for the TEG used. Following cold start, the power converter boosts the TEG voltage to 1.5 V DV_{DD} within 200 ms. AV_{DD} ramps up slowly to 1.2 V, as shown in FIG. 8.

Referring back to FIG. 7, the slow ramp up of AV_{DD} ensures settling of initial VREFL (low reference voltage) and VREFH (high reference voltage), generated by DV_{DD}-powered leakage-based voltage references, before AFE 107 starts operation and initializes the PWLLs. With the incidence of ECG pulses, VREFL and VREFH self-train and adapt to the subject motion. The average power consumption of the AFE is, for example 504 nW, and that of the digital blocks 108 for adaptive threshold generation and heartbeat detection is for example, 92 nW. In some examples, a majority of the SoC average power consumption (e.g., 4.6 μW) is spent by the Tx local oscillator (LO) 106a, which remains always on and operates at, for example, greater than 8 MHz. The transmitter (Tx) power amplifier (PA) 106c is merely enabled when the heartbeat is detected and draws remaining available power from the harvester to transmit to the antenna. Although this higher instantaneous power consumption of PA 106c increases the minimum operational power requirement of the SoC, the average PA power consumption is negligible due to extremely small duty-cycle of the HB (heart beat) pulse and does not affect the average SoC power consumption.

FIG. 8 illustrates a set of plots 800, 820, 830, and 840 that show measured start-up transients of power rails during cold-start, initialization of AFE 107 and VREFs; and harvested power utilization at minimum TEG voltage, in accordance with embodiments of the present disclosure. FIG. 8 shows the utilization of harvested power by the SoC blocks when all of them are operational (e.g., HB is high) at the minimum TEG voltage of, for example 20mV, and a maximum input power of, for example 20 μW, to the harvester.

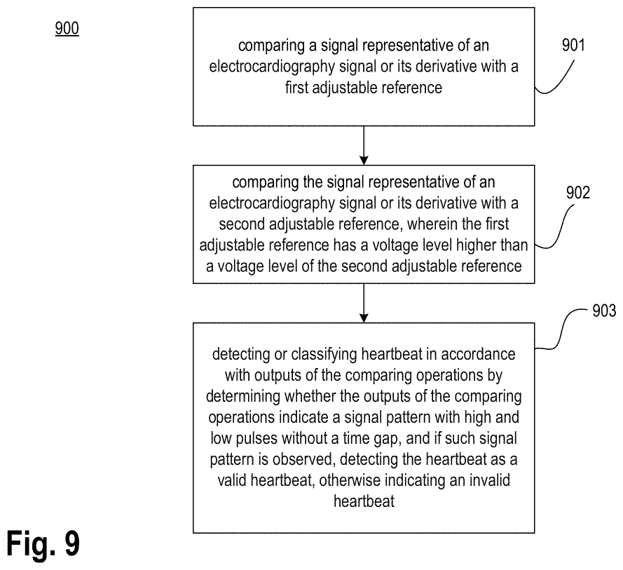

FIG. 9 illustrates flowchart 900 of a method for determining a heartbeat, in accordance with some embodiments. While blocks are illustrated in a particular order, the order can be modified. For example, some blocks can be performed before others while some blocks can be performed in parallel. The various blocks here can be performed by hardware and/or software.

At block 901, comparator 108a compares a signal representative of an ECG or its derivative with a first adjustable reference VREFH. The ECG or its derivative is generated by AFE 107. In some embodiments, first adjustable reference VREFH is generated by a PWD scheme comprising charge pump CP_{HI} as discussed with reference to FIG. 3. In some embodiments, first adjustable reference VREFH is adjusted by hardware (e.g., CP_{HI} and PWD 301) and/or software (e.g., software to control capacitance C1).

At block 902, comparator 108b compares the ECG signal or its derivative with a second adjustable reference VREFL. In some embodiments, second adjustable reference VREFL is generated by a PWD scheme comprising charge pump CP_{LO} as discussed with reference to FIG. 3. In some embodiments, second adjustable reference VREFL is adjusted by hardware (e.g., CP_{LO} and PWD 302) and/or software (e.g., software to control capacitance C2). In various embodiments, the first adjustable reference VREFH has a voltage level higher than the voltage level of the second adjustable reference VREFL

At block 903, logic 108e detects or classifies the HB in accordance with the outputs PW_{H} and PW_{L} from comparators 108a and 108b, respectively. Here, TPW1(n) and TPW2(n) are the nth pulse widths of the corresponding signals. In some embodiments, logic 108e determines whether the outputs TPW1(n) and TPW2(n) of the comparing operations indicate a signal pattern with high and low pulses without a time gap. If such signal pattern is observed, logic 108e determines that the heartbeat is a valid heartbeat, otherwise logic 108e indicates an in lid heartbeat.

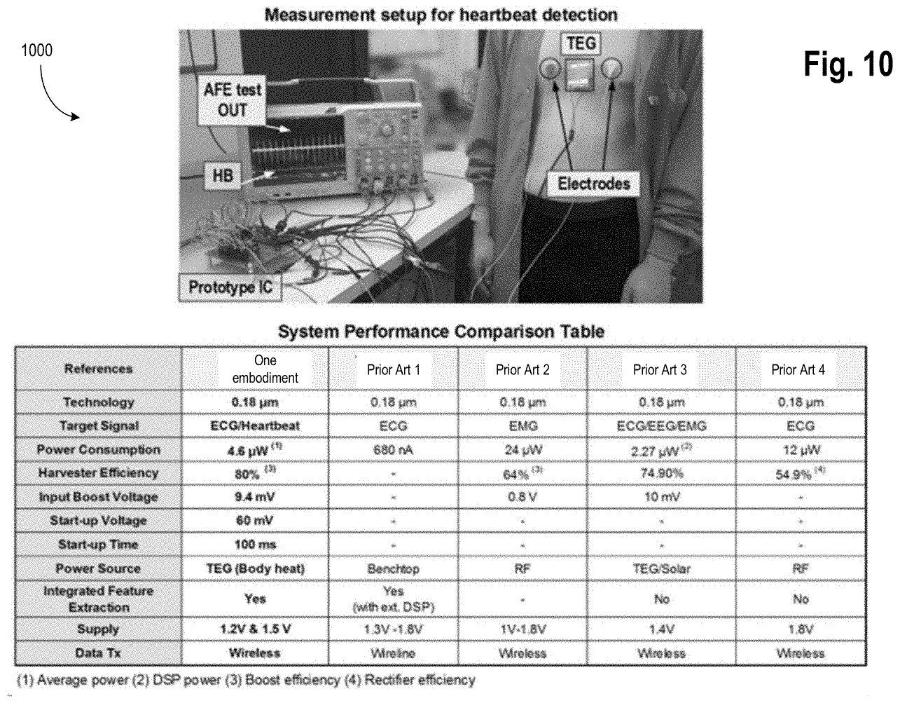

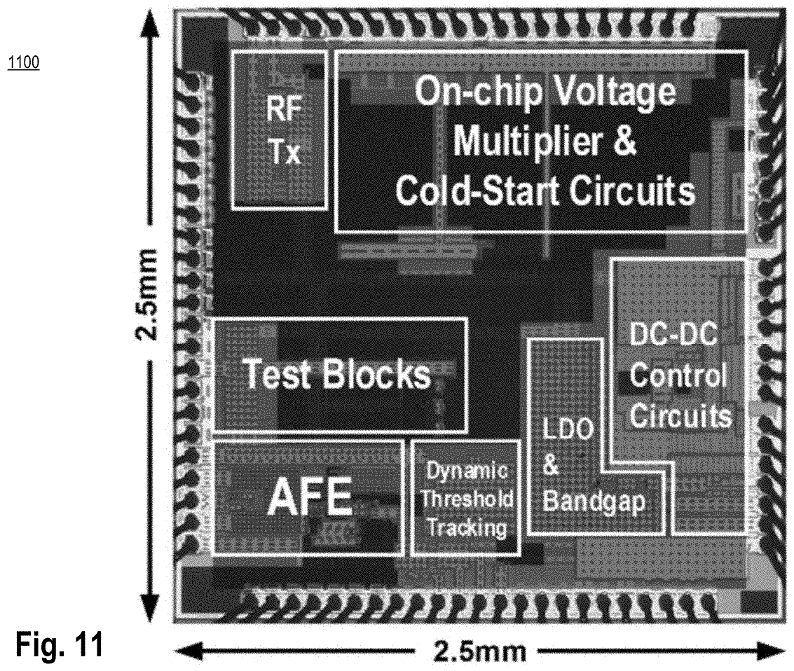

A comparison of the system performance with related approaches and the ECG measurement setup 1000 are shown in FIG. 10. The prototype is implemented in a 0.18 μm CMOS and with 6.25 mm² die area; a die photo 1100 is shown in FIG. 11. The self-sustaining system demonstrates an energy-autonomous heartbeat detection system powered entirely and continuously by body heat.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional elements.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

While the disclosure has been described in conjunction with specific embodiments thereof, many alternatives, modifications and variations of such embodiments will be apparent to those of ordinary skill in the art in light of the foregoing description. The embodiments of the disclosure are intended to embrace all such alternatives, modifications, and variations as to fall within the broad scope of the appended claims.

In addition, well-known power/ground connections to integrated circuit (IC) chips and other components may or may not be shown within the presented figures, for simplicity of illustration and discussion, and so as not to obscure the disclosure. Further, arrangements may be shown in block diagram form in order to avoid obscuring the disclosure, and also in view of the fact that specifics with respect to implementation of such block diagram arrangements are highly dependent upon the platform within which the present disclosure is to be implemented (i.e., such specifics should be well within purview of one skilled in the art). Where specific details (e.g., circuits) are set forth in order to describe example embodiments of the disclosure, it should be apparent to one skilled in the art that the disclosure can be practiced without, or with variation of, these specific details. The description is thus to be regarded as illustrative instead of limiting.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

The following examples are provided with reference to various embodiments. These examples can be combined in any suitable manner.

Example 1: An apparatus comprising: a first electrical loop comprising a first comparator to receive a signal representative of an electrocardiography signal or its derivative and to compare it a first adjustable reference; a second electrical loop comprising a second comparator to receive the signal representative of an electrocardiography signal or its derivative and to compare it with a second adjustable reference, wherein the first adjustable reference has a voltage level higher than a voltage level of the second adjustable reference; and logic to detect or classify a heartbeat in accordance with an output of the first and second comparators.

Example 2: The apparatus of example 1, wherein the first electrical loop comprises a first pulse width detector (PWD) coupled to an output of the first comparator.

Example 3: The apparatus of example 2, wherein the first PWD is to generate a first Up and/or Down signal indicative of a first pulse width of the output of the first comparator with reference to a third reference.

Example 4: The apparatus of example 3, wherein the third reference is adjustable by software and/or hardware.

Example 5: The apparatus of example 3 comprises: a first capacitor; and a first charge pump coupled to the first PWD, wherein the first charge pump is to sink or source current into the first capacitor in accordance with the first Up and/or Down signals, wherein a change on the first capacitor translates to the first adjustable reference.

Example 6: The apparatus of example 2, wherein the second electrical loop comprises a second PWD coupled to an output of the second comparator.

Example 7: The apparatus of example 6, wherein the second PWD is to generate second Up and/or Down signals indicative of a second pulse width of the output of the second comparator with reference to a fourth reference.

Example 8: The apparatus of example 7, wherein the fourth reference is adjustable by software and/or hardware.

Example 9: The apparatus of example 7 comprises: a second capacitor; and a second charge pump coupled to the first PWD, wherein the second charge pump is to sink or source current into the second capacitor her accordance with the second Up and/or Down signals, wherein a change on the second capacitor translates to the second adjustable reference.

Example 10: The apparatus of example 1, wherein the logic comprises: circuitry to determine whether the first and second outputs indicate a signal pattern with high and low pulses without a time gap, and if such signal pattern is observed, detect the heartbeat as a valid heartbeat, otherwise indicate an invalid heartbeat.

Example 11: A method comprising: comparing a signal representative of an electrocardiography signal or its derivative with a first adjustable reference; comparing the signal representative of an electrocardiography signal or its derivative with a second adjustable reference, wherein the first adjustable reference has a voltage level higher than voltage level of the second adjustable reference; and detecting or classifying heartbeat in accordance with outputs of the comparing operations.

Example 12: The method of example 8 comprising determining whether the outputs of the comparing operations indicate a signal pattern with high and low pulses without a time gap, and if such signal pattern is observed, detecting the heartbeat as a valid heartbeat, otherwise indicating an invalid heartbeat.

Example 13: A system-on-chip comprising: a first circuitry to generate a first power supply voltage on a first rail and a second power supply voltage on a second rail, wherein the first power supply voltage is generated from energy harvested from a living body, and wherein the second power supply is generated using the first power supply voltage as input supply; a second circuitry to detect an ECG signal from the living body, wherein the second circuitry operates on the second power supply; and a third circuitry to determine a heartbeat from the ECG signal, wherein the third circuitry operates on the first power supply.

Example 14: The system-on-chip of example 13 comprising a fourth circuitry to transmit the heartbeat via an antenna.

Example 15: The system-on-chip of example 13, wherein the first circuitry comprises: a DC-DC boost converter to generate the first power supply voltage, and a low dropout regulator to generate the second power supply voltage from the first power supply voltage.

Example 16: The system-on-chip of example 13, wherein the second circuitry comprises: a low-noise amplifier to receive signals from two sensors on the living body; a differentiator coupled to the low-noise amplifier; and a filter coupled to the differentiator, wherein an output of the filter is the ECG signal.

Example 17: The system-on-chip of example 13, wherein the third circuitry comprises: a first electrical loop comprising a first comparator to receive a signal representative of an electrocardiography signal or its derivative and to compare it with a first adjustable reference; a second electrical loop comprising a second comparator to receive the signal representative of an electrocardiography signal or its derivative and to compare it with a second adjustable reference, wherein the first adjustable reference has a voltage level higher than a voltage level of the second adjustable reference; and logic to detect or classify a heartbeat in accordance with an output of the first and second comparators.

Example 18: The system-on-chip of example 17, wherein the first electrical loop comprises a first pulse width detector (PWD) coupled to an output of the first comparator, wherein the first PWD is to generate a first Up and/or Down signal indicative of a first pulse width of the output of the first comparator with reference to a third reference, wherein the third reference is adjustable by software and/or hardware.

Example 19: The system-on-chip of example 18 comprises: a first capacitor; and a first charge pump coupled to the first PWD. wherein the first charge pump is to sink or source current into the first capacitor in accordance with the first Up and/or Down signals, wherein a change on the first capacitor translates to the first adjustable reference.

Example 20: The system-on-chip of example 18, wherein the second electrical loop comprises a second PWD coupled to an output of the second comparator, wherein the second PWD is to generate second Up and/or Down signals indicative of a second pulse width of the output of the second comparator with reference to a fourth reference, wherein the fourth reference is adjustable by software and/or hardware.

Example 21: The system-on-chip of example 20 comprises: a second capacitor; and a second charge pump coupled to the first PWD, wherein the second charge pump is to sink or source current into the second capacitor in accordance with the second Up and/or Down signals, wherein a change on the second capacitor translates to the second adjustable reference.

Example 22: The system-on-chip of example 17, wherein the logic comprises: fifth circuitry to determine whether the first and second outputs indicate a signal pattern with high and low pulses without a time gap, and if such signal pattern is observed, detect the heartbeat as a valid heartbeat, otherwise indicate an invalid heartbeat.

An abstract is provided that will allow the reader to ascertain the nature and gist of the technical disclosure. The abstract is submitted with the understanding that it will not be used to limit the scope or meaning of the claims. The following claims are hereby incorporated into the detailed description, with each claim standing on its own as a separate embodiment.

What is claimed is:

1. An apparatus comprising:
a first electrical loop comprising a first comparator to receive a signal representative of an electrocardiography signal or its derivative and to compare it with a first adjustable reference;
a second electrical loop comprising a second comparator to receive the signal representative of an electrocardiography signal or its derivative and to compare it with a second adjustable reference, wherein the first adjustable reference has a voltage level higher than a voltage level of the second adjustable reference; and
logic to detect or classify a heartbeat in accordance with an output of the first and second comparators.

2. The apparatus of claim 1, wherein the first electrical loop comprises a first pulse width detector (PWD) coupled to an output of the first comparator.

3. The apparatus of claim 2, wherein the first PWD is to generate a first Up and/or Down signal indicative of a first pulse width of the output of the first comparator with reference to a third reference.

4. The apparatus of claim 3, wherein the third reference is adjustable by software and/or hardware.

5. The apparatus of claim 3 comprises:
a first capacitor; and
a first charge pump coupled to the first PWD, wherein the first charge pump is to sink or source current into the first capacitor in accordance with the first Up and/or Down signals, wherein a change m the first capacitor translates to the first adjustable. reference.

6. The apparatus of claim 2, wherein the second electrical loop comprises a second PWD coupled to an output of the second comparator.

7. The apparatus of claim 6, wherein the second PWD is to generate second Up and/or Down signals indicative of a second pulse width of the output of the second comparator with reference to a fourth reference.

8. The apparatus of claim 7, wherein the fourth reference is adjustable by software and/or hardware.

9. The apparatus of claim 7 comprises:
a second capacitor; and
a second charge pump coupled to the first PWD, wherein the second charge pump is to sink or source current into the second capacitor in accordance with the second Up and/or Down signals, wherein a change on the second capacitor slates to the second adjustable reference.

10. The apparatus of claim 1, wherein the logic comprises:
circuitry to determine whether the first and second outputs indicate a signal pattern with high and low pulses without a time gap, and if such signal pattern is observed, detect the heartbeat as a valid heartbeat, otherwise indicate an invalid heartbeat.

11. A method comprising:
comparing a signal representative of an electrocardiography signal or its derivative with a first adjustable reference;
comparing the signal representative of an electrocardiography signal or its derivative with a second adjustable reference, wherein the first adjustable reference has a voltage level higher than a voltage level of the second adjustable reference; and
detecting or classifying heartbeat in accordance with outputs from the comparing.

12. The method of claim 8 comprising:
determining whether the outputs from the comparing indicate a signal pattern with high and low pulses without a time gap, and if such signal pattern is observed, detecting the heartbeat as a valid heartbeat, otherwise indicating an invalid heartbeat.

13. A system-on-chip comprising:
a first circuitry to generate a first power supply voltage on a first rail and a second power supply voltage on a second rail, wherein the first power supply voltage is generated from energy harvested from a living body, and wherein the second power supply is generated using the first power supply voltage as input supply;
a second circuitry to detect an ECG signal from the living body, wherein the second circuitry operates on the second power supply; and
a third circuitry to determine a heartbeat from the ECG signal, wherein the third circuitry operates on the first power supply.

14. The system-on-chip of claim 13 comprising a fourth circuitry to transmit the heartbeat via an antenna.

15. The system-on-chip of claim 13, wherein the first circuitry comprises:
a DC-DC boost converter to generate the first power supply voltage, and
a low dropout regulator to generate the second power supply voltage from the first power supply voltage. 16, The system-on-chip of claim 13, wherein the second circuitry comprises:
a low-noise amplifier to receive signals from two sensors on the living body;
a differentiator coupled to the low-noise amplifier; and
a filter coupled to the differentiator, wherein an output of the filter is the ECG signal.

17. The system-on-chip of claim 13, wherein the third circuitry comprises:
a first electrical loop comprising a first comparator to receive a signal representative of an electrocardiography signal or its derivative and to compare it with a first adjustable reference;
a second electrical loop comprising a second comparator to receive the signal representative of an electrocardiography signal or its derivative and to compare it with a second adjustable reference, wherein the first adjustable reference has a voltage level higher than a voltage level of the second adjustable reference; and
logic to detect or classify a heartbeat in accordance with an output of the first and second comparators.

18. The system-on-chip of claim 17, wherein the first electrical loop comprises a first pulse width detector (PWD) coupled to an output of the first comparator, wherein the first PWD is to generate a first Up and/or Down signal indicative of a first pulse width of the output of the first comparator with reference to a third reference, wherein the third reference is adjustable by software and/or hardware.

19. The system-on-chip of claim 18 comprises:
a first capacitor; and
a first charge pump coupled to the first PWD, wherein the first charge pump is to sink or source current into the first capacitor in accordance with the first Up and/or Down signals, wherein a change on the first capacitor translates to the first adjustable reference.

20. The system-on-chip of claim 18, wherein the second electrical loop comprises a second PWD coupled to an output of the second comparator, wherein the second PWD is to generate second Up and/or Down signals indicative of a second pulse width of the output of the second comparator with reference to a fourth reference, wherein the fourth reference is adjustable by software and/or hardware.

21. The system-on-chip of claim 20 comprises:
a second capacitor; and
a second charge pump coupled to the first PWD, wherein the second charge pump is to sink or source current into the second capacitor in accordance with the second Up and/or Down signal, wherein a change on the second capacitor translates to the second adjustable reference.

22. The system chip of claim 17, wherein the logic comprises:
fifth circuitry to determine whether the first and second outputs indicate a signal pattern with high and low pulses without a time gap, and if such signal pattern is observed, detect the heartbeat as a valid heartbeat, otherwise indicate an invalid heartbeat.
